(19) United States
(12) Patent Application Publication
(10) Pub. No.: US 2019/0148023 A1
(43) Pub. Date: May 16, 2019

(54) Machine-Learned Epidemiology

(71) Applicant: Google LLC, Mountain View, CA (US)

(72) Inventors: Adam Sadilek, San Jose, CA (US); Evgeniy Gabrilovich, Saratoga, CA (US)

(21) Appl. No.: 15/869,215

(22) Filed: Jan. 12, 2018

Related U.S. Application Data
(60) Provisional application No. 62/587,297, filed on Nov. 16, 2017.

(51) Int. Cl. G16H 50/80; G06N 99/00; G06F 17/30

(52) U.S. Cl. CPC G16H 50/80; G06F 17/30864; G06N 99/005

(57) ABSTRACT

The present disclosure provides systems and methods that leverage machine-learned models in conjunction with online data to monitor and detect the spread of a disease, such as, for example, a communicable illness. In one example, a computing system can include or otherwise leverage a machine-learned disease detection model. The computing system can input search engine data and, optionally, location data respectively associated with a first plurality of users into the machine-learned disease detection model. The computing system can receive identification of a second plurality of users predicted to have the disease as an output of the machine-learned disease detection model. The second plurality of users can be a subset of the first plurality of users. The computing system can identify one or more locations associated with elevated levels of the disease based at least in part on the location data respectively associated with at least the second plurality of users.

### RELATED APPLICATION DATA

This application claims the benefit of and priority to U.S. Provisional Patent Application No. 62/587,297, filed on Nov. 16, 2017. U.S. Provisional Patent Application No. 62/587,297 is hereby incorporated by reference in its entirety.

### FIELD

The present disclosure relates generally to machine learning and epidemiology. More particularly, the present disclosure relates to the use of machine-learned models to identify locations associated with elevated levels of a disease and/or individuals at risk from disease.

### BACKGROUND

Tracking, monitoring, and preventing the spread of diseases is difficult for a variety of reasons, including but not limited to a lack of real-time data and a limit in the availability of data. In recent years, the advent of computational epidemiology has improved the speed and accuracy with which diseases can be tracked and monitored. However, work in computational epidemiology to date has been limited by coarseness and lack of timeliness of observational data. Most existing models are based on government-collected statistics that are several years delayed, aggregated, expensive to curate, and cover only limited jurisdictions.

One example of a disease for which improved tracking, monitoring, and spread prevention would be beneficial is foodborne illness. A major challenge in monitoring and reducing foodborne illness is early identification of these outbreaks in real time. While public health authorities do receive and respond to complaints about specific restaurants, rates of foodborne illness are drastically underreported. Even when foodborne illness is reported to a local health authority, time from initial report to confirmed serotyping of a pathogen can take 2-3 weeks, which drastically slows the response time. Furthermore, consumer driven reports are themselves biased and noisy. Even the most sophisticated systems, such as the Foodborne Diseases Active Surveillance Network (FoodNet), have a significant time lag in processing and reporting information. Foodborne illnesses have enormous medical implications, impacting nearly 50 million Americans every year, leading to 3,000 deaths. However, the Centers for Disease Control and Prevention (CDC) has categorized food safety as one of its seven winnable battles, meaning that while it currently has substantial negative impacts on the health of many, there is great potential to reduce the associated morbidity and mortality.

More generally, approaches that lead to real-time identification of illness while the outbreak is still occurring can prevent additional cases and lead to far quicker resolution. As one example, malaria is a vector borne illness that has 212 million cases per year resulting in approximately 400 thousand deaths. The number of deaths is one extreme metric, but even survivors go through unimaginable suffering and long-term disability. While malaria currently has substantial negative impacts on the health of many, there is great potential to reduce the associated morbidity and mortality with preventative or earlier treatment or combative procedures/interventions. Thus, the real-time identification of malaria outbreaks can help prevent the spread of malaria and reduce the number of cases and deaths per year attributable to the disease.

Thus, improved systems for tracking, monitoring, and preventing the spread of various different diseases can provide both significant improvements in public health while also reducing the resources required to treat or otherwise respond to the diseases.

### SUMMARY

Aspects and advantages of embodiments of the present disclosure will be set forth in part in the following description, or can be learned from the description, or can be learned through practice of the embodiments.

One example aspect of the present disclosure is directed to a computing system. The computing system includes one or more processors. The computing system includes a machine-learned disease detection model. The computing system includes one or more non-transitory computer-readable media that collectively store instructions that, when executed by the one or more processors, cause the one or more processors to perform operations. The operations include obtaining search engine data and location history data respectively associated with a first plurality of users. The operations include inputting at least the search engine data into a machine-learned disease detection model. The operations include receiving, as an output of the machine-learned disease detection model, identification of a second plurality of users predicted to have the disease. The second plurality of users is a subset of the first plurality of users. The operations include identifying one or more locations associated with elevated levels of the disease based at least in part on the location history data respectively associated with at least the second plurality of users.

Another example aspect of the present disclosure is directed to a computer-implemented method to identify locations associated with a disease. The method includes obtaining, by one or more computing devices, search engine data and location data respectively associated with a first plurality of users. The method includes inputting, by the one or more computing devices, at least the search engine data into a machine-learned disease detection model. The method includes receiving, by the one or more computing devices as an output of the machine-learned disease detection model, identification of a second plurality of users predicted to have the disease. The second plurality of users is a subset of the first plurality of users. The method includes identifying, by the one or more computing devices, one or more locations associated with the disease based at least in part on the location data respectively associated with at least the second plurality of users.

Another example aspect of the present disclosure is directed to one or more non-transitory computer-readable media that collectively store instructions that, when executed by one or more processors, cause the one or more processors to perform operations. The operations include obtaining location history data respectively associated with a first plurality of users. The operations include inputting the location history data into a machine-learned disease detection model. The operations include receiving, as an output of the machine-learned disease detection model, identification of a second plurality of users predicted to have the disease. The second plurality of users is a subset of the first plurality of users. The operations include identifying one or more locations associated with the disease based at least in part on the location history data respectively associated with at least the second plurality of users.

Another example aspect of the present disclosure is directed to a computer-implemented method. The method includes receiving, by one or more computing devices, user data. The method includes determining, by the one or more computing devices, correlations between the user data and one or both of disease or location. The method includes storing, by the one or more computing devices, the correlations. The method includes iteratively updating, by the one or more computing devices, the correlations upon receipt of new user data to form a predictive disease detection model. The method includes using, by the one or more computing devices, the predictive disease detection model to determine a predicted causation of a disease outbreak.

Another example aspect of the present disclosure is directed to a computing system that includes one or more processors and a machine-learned disease detection model implemented by the one or more processors. The machine-learned disease detection model was trained by performing operations. The operations include, for each of a plurality of positive training example search queries, automatically inferring a disease label to apply to such positive training example search query based at least in part on content included in a web page result selected by a user after entering such positive training example search query. The operations include sampling a set of additional search queries to obtain negative training example search queries. The operations include training the machine-learned disease detection model to differentiate between the positive training example search queries and the negative training example search queries.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1 depicts a block diagram of an example computing system according to example embodiments of the present disclosure.

FIG. 2 depicts a block diagram of an example processing pipeline according to example embodiments of the present disclosure.

FIG. 3 depicts a flow chart diagram of an example method to perform disease detection according to example embodiments of the present disclosure.

Reference numerals that are repeated across plural figures are intended to identify the same features in various implementations.

### DETAILED DESCRIPTION

### Overview

Example aspects of the present disclosure are directed to systems and methods that include or otherwise leverage machine-learned models in conjunction with online data to monitor and detect the spread of a disease, such as, for example, a communicable and/or infectious illness. In one example, a computing system can include or otherwise leverage a machine-learned disease detection model. The computing system can input search engine data and, optionally, location data (e.g., location history data) respectively associated with a first plurality of users into the machine-learned disease detection model. The computing system can receive identification of a second plurality of users predicted to have the disease as an output of the machine-learned disease detection model. The second plurality of users can be a subset of the first plurality of users. The computing system can identify one or more locations associated with elevated levels of the disease based at least in part on the location data respectively associated with at least the second plurality of users. The computing system can provide one or more alerts to users, disease abatement systems, and/or appropriate governmental authorities regarding the identified locations. Thus, the present disclosure provides improved systems for tracking, monitoring, and preventing the spread of various different diseases. In particular, the systems and methods described herein can identify in real time locations associated with an elevated level of a disease and/or individuals at risk from disease based on online data such as search engine data, all in a privacy preserving way. As such, the systems and methods of the present disclosure can provide both significant improvements in public health while also reducing the resources required to treat or otherwise respond to the diseases. In particular, the systems and methods of the present disclosure can enable timely prevention of spread of disease by leveraging precise data-driven health inferences and predictions.

More particularly, a computing system can work to identify locations associated with an elevated level of a disease. A disease can be a communicable disease or illness; an infectious disease; a transmissible disease; a disease caused by a pathogen such as bacterial pathogens, viral pathogens, fungi or protozoa pathogens; an allergic disease; and/or other forms of diseases. Example diseases include malaria, Ebola, cholera, influenza, Lyme disease, varicella, variola, dengue fever, foodborne illness, etc.

According to an aspect of the present disclosure, to identify the locations associated with elevated levels of the disease, the computing system can obtain data associated with a first plurality of users. For example, the data can include search engine data and/or location data. The data can also include additional feature data, as described further below. For example, the location data can include current location data that describes current location of users and/or location history data that describes historical locations of users in the past. For example, the location history data can include sets of location (e.g., in the form of geo-coordinates) and time (e.g., in the form of a timestamp). For example, a user's device (e.g., smartphone) can periodically (e.g., once per minute) communicate its current location (e.g., as determined by a GPS, Wi-Fi triangulation, cell tower triangulation, etc.) to a centralized server which can generate and store a log of such information. Alternatively or additionally, the user's location can be derived from the search query and/or IP address of the device. Such location can optionally be stored in the log. In some implementations, the first plurality of users is not a specifically defined set of users but instead refers to the general population as a whole, or at least the general population for which the corresponding data (e.g., search engine data) is available.

In some implementations, the computing system can obtain the search engine data and/or location data from or in the form of logs stored in one or more databases associated with a search engine service and/or location service. As examples, the search engine data can include data descriptive of search queries; scrolling actions; result selection actions; time spent on user-selected results; content of user-selected results; and/or other search engine information.

Thus, as an example, a user can enter a search query, receive the search results, scroll past a first result to a second result, select the second result, spend five minutes reading the second result, return to the search result page, select a third result, and spend 20 minutes reading the third result. In such example, each of the search query, the scrolling action, result selection, time spent on selected results, and content of selected results can be recorded in a log. As will be discussed further below, this information can be used (e.g., in real time) to determine whether the user is suffering from a particular disease. In particular, the feature of content of selected results can be highly important to assist is disambiguating a generic search query (e.g., symptom-based search query) to a specific disease. As an example, a user might search for “stomach ache,” which could correspond to a number of different maladies. If the user scrolls past a first result that describes heartburn to a second result that includes content directed to foodborne illnesses (“food poisoning”), this can be a highly useful signal that the user is suffering from foodborne illness rather than heartburn.

Thus, the search engine data or other user data does not need to be explicitly about or otherwise directly identify a particular disease. Instead, for example, the data can generally describe a number of related symptoms, indicators, or other signals or data points suggestive of a disease. When these related items are correlated in time and/or location they may suggest that one or more users are suffering from a disease which can cause the symptoms, indicators, or other signals described by the user data. For example, the search queries “sneezing” and “runny nose” do not explicitly identify the disease of “seasonal allergies.” However, when combined with other contextual signals or indicators such as, for example, search data indicating that a user selected and read a search result about allergic reactions and/or transaction data indicating that the user purchased allergy medications online, these signals can collectively indicate that the user is suffering from seasonal allergies.

Further to the descriptions above, a user may be provided with controls allowing the user to make an election as to both if and when systems, programs or features described herein may enable collection of user information (e.g., information about a user's search engine queries or other search engine data, location data, online transaction data, a user's preferences, or other user data), and if the user is sent content or communications from a server. In addition, as will be described further below, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over what information is collected about the user, how that information is used, and what information is provided to the user.

In particular, in some implementations, the computing system can remove personal information from the obtained data so that information such as specific user identities or user identifiers is not included in the data. For example, in some implementations, the computing system can respectively assign a unique and non-personally identifying identifier to the search engine data for each of the first plurality of users and then remove any personal information from the search engine data. As examples, the unique and non-personally identifying identifier can include a random number, a result of a hash function applied to a user identifier, or some other obfuscated or encrypted identifier which cannot be reverted to obtain the user identifier. Likewise, the computing system can respectively assign the same unique and non-personally identifying identifier to the location data for each of the first plurality of users and then remove personal information from the location data.

Thus, following a privacy protection process, the computing system can obtain sets of search engine data that are respectively connectable to sets of location data by way of a shared unique and non-personally identifying identifier. However, these sets of data contain no personal information such as specific user identities. As such, the data is anonymous, no user-level labels are stored, and, as will be described further below, the computation aggregates all data at the location level.

According to another aspect of the present disclosure, the computing system can input at least the search engine data into a machine-learned disease detection model. For example, the computing system can input each set of search engine data for each user into the model individually (e.g., on a per-user basis). In some implementations, in addition or alternatively to the search engine data, the computing system can input the location data (e.g., location history data) into the machine-learned disease detection model.

In some implementations, additional feature data can also be input into the machine-learned disease detection model. As examples, the additional feature data can include step count data for a user (e.g., that is generated by a computing device that is worn by the user); heart rate data for a user (e.g., that is generated by a computing device that is worn by the user); weather condition data (e.g., current and/or historical weather condition data such as temperature, humidity, etc. for a number of different location); user biometrics data; user health data; mosquito density data; water quality data; and/or various other types of features including any features that have been shown to be predictors of and/or correlated to the disease being investigated.

As another example, the additional feature data can include other online data for users in addition or alternatively to the search engine data. For example, the additional online data can include online transaction data. For example, online transaction data indicative that a user has ordered allergy medication, water purification pills, or other disease remedies can be indicative that the user is suffering from a disease.

The computing system can receive, as an output of the machine-learned disease detection model, identification of a second plurality of users predicted to have the disease. The second plurality of users can be a subset of the first plurality of users. Thus, in some implementations, the computing system can input the search engine data, location data (e.g., location history data), and/or other feature data associated with a user into the machine-learned disease detection model and can receive a prediction from the machine-learned disease detection model as to whether such user is suffering from the disease. In some implementations, the prediction can indicate whether the user is currently suffering from the disease and/or whether the user suffered from the disease in the past and, if so, at what time in the past. In some implementations, the prediction can include a probability and/or confidence score associated with the prediction. In some implementations, the prediction can include a predicted time at which the user acquired the disease.

In some implementations, the computing system can include multiple different machine-learned disease detection models that have been respectively trained to detect multiple different diseases (e.g., a foodborne illness model, a malaria model, a dengue fever model, etc.). The available data can be separately input into each model in parallel. In other implementations, a single machine-learned model can be trained to provide a multi-class classification prediction with respect to the multiple different diseases. As examples, each of the one or more machine-learned disease detection models can be or include one or more of a support vector machine, a deep neural network, a log-linear model, and/or other machine-learned models. For example, the log-linear model can be or include a log-linear maximum entropy model.

According to another aspect of the present disclosure, the computing system can identify one or more locations associated with elevated levels of the disease based at least in part on the location data (e.g., location history data) respectively associated with at least the second plurality of users. Thus, for example, the computing system can analyze current and/or past locations associated with the second plurality of users to identify locations that exhibit an increased or otherwise elevated number of persons with the disease. For example, clustering techniques can be performed on the location data to identify clusters at a particular location.

In some implementations, the computing system can identify the one or more locations associated with elevated levels of the disease based at least in part on the location data (e.g., location history data) respectively associated with both the second plurality of users and a third plurality of users. The third plurality of users can include those users that are included in the first plurality of users but not the second plurality of users.

As one example, in some implementations, the computing system can identify the one or more locations from a plurality of candidate locations. For example, the candidate locations can be geographical areas (e.g., map tiles that correspond to certain areas of the Earth, states, counties, cities, neighborhoods, property parcels, or other divisions of geographic area); establishments (e.g., restaurants); buildings; floors within a building; certain transit vehicles (e.g., a particular airplane or bus); geographic coordinates; or other representations of location.

In some implementations, to identify the one or more locations associated with the disease from the one or more candidate locations, the computing system can determine, for each of the plurality of candidate locations, a ratio of a first number of the second plurality of users that have visited the candidate location within a threshold amount of time to a second number of the third plurality of users that have visited the candidate location within the threshold amount of time. For example, in some instances, the threshold amount of time can be determined based on an incubation period associated with the disease being investigated. Thus, in some implementations, the computing system can determine, for each candidate location, a ratio of the number of visitors predicted to be suffering from the disease versus the number of visitors predicted to not be suffering from the disease. In other implementations, the computing system can determine, for each candidate location, a ratio of the number of visitors predicted to be suffering from the disease versus the total number of visitors (e.g., inclusive of all visitors regardless of disease status).

The computing system can identify one or more of the candidate locations as being associated with the disease based at least in part on the respective ratios determined for the plurality of candidate locations. As one example, the computing system can compare each respective ratio to a threshold value to determine whether the corresponding candidate location is associated with the disease. For example, if the ratio is greater than the threshold value, then the candidate location can be designated as being associated with elevated levels of the disease.

In some implementations, the threshold value used for each candidate location can be different or otherwise specific to such candidate location. For example, the threshold value for a candidate location can be based on a baseline ratio that has been demonstrated at such candidate location in the past (e.g., an average ratio demonstrated over time). As another example, the threshold value used for each candidate location can be dynamically determined based on overall levels of disease incidence seen in the populace as a whole over the threshold amount of time. Thus, in such implementations, a candidate location will not be identified as corresponding to elevated levels just because an illness is “going around” and the candidate location sees a rise in in its ratio, but instead will only be identified as corresponding to elevated levels if the rise in its ratio outpaces rises in ratios observed for other locations.

As another example, in some implementations, the computing system can identify the one or more locations associated with the disease by inputting the location history data respectively associated with the second plurality of users into a machine-learned location detection model. The computing system can receive identification of one or more visited locations associated with the second plurality of users. In some implementations, the visited locations can be designated as associated with the disease. However, in other implementations, the visited locations identified by the machine-learned location detection model can simply be used to compute the ratios described above for various locations. As such, in some implementations, the computing system can input the location history data for all users (e.g., including the third plurality of users) to simply receive indications of locations that each user has visited (e.g., for the purpose of computing ratios as described above).

In some implementations, the computing system can identify one or more additional users that are predicted to be suffering from the disease. For example, these additional users can be predicted to be suffering from the disease based on their respective location data (e.g., location history data). As one example, twenty people who drank the water from a particular location performed searches indicative of cholera. The particular location may be identified as being associated with an outbreak of cholera based on such search data, as described above. Further, the computing system can identify additional users that, based on their respective location histories, are predicted to be suffering from cholera. For example, additional users that visited the particular location can be identified, warned, and/or afforded additional attention in some manner. Thus, the computing system can proactively identify additional users that may be suffering from a disease (e.g., prior to conclusion of the incubation period), thereby leading to proactive treatment of the disease. Automated systems may be involved in such proactive treatment. For example, medicines may be dispatched automatically to the additional users. They may also receive automated notifications (e.g. an email and/or text message) advising them that they should see a doctor at their earliest convenience.

More generally, in some implementations, the machine-learned disease detection model can detect trends in data such as online data or other user data that indicates that a group of users has some correlation. For example, the disease detection model can identify a group of users that have some activity (e.g., search engine interactions, location visits, etc.) that indicate that they have some commonality, particular with respect to a disease and/or its symptoms. Based on these correlations among users and their corresponding data, the machine-learned disease detection model and/or additional system components can provide an indication of locations that have exhibited, are current exhibiting, and/or are predicted to exhibit elevated levels of a disease.

According to another aspect of the present disclosure, the computing system can provide one or more alerts to users, disease abatement systems, manufacturers of products and services to remediate the disease, producers and distributors of medicines, hospitals and emergency response providers, and/or appropriate governmental authorities regarding the locations that have been identified as having elevated levels of the disease or predicted to have elevated levels of the disease. As one example, an alert can be sent to a user (e.g., a user that regularly visits or is planning to visit the location) that informs the user to avoid the location.

As another example, an alert can be sent to a disease abatement system. For example, if the disease is a mosquito-borne illness, the computing system can alert (e.g., automatically dispatch) a mosquito spraying vehicle to spray the identified locations.

As another example, an alert can be sent to a producer of a medicine that treats the disease. For example, if the disease is malaria, the computing system can alert a producer of anti-malarial medicines about a predicted current or upcoming outbreak of malaria at a certain location. This can enable the producer of the anti-malarial medicine to change their manufacturing operations to better combat the outbreak of malaria. For example, the producer can change their manufacturing operations to generate additional pills and can change their logistics operations to ensure that the additional pills are transported to the identified location at the appropriate time, actions which would otherwise require a delay until after the outbreak was apparent.

As another example, an alert can be sent which results in or enables additional interventions such as, for example, targeted distribution of vaccines, drugs, vector repellent, education about disease avoidance, vector netting and/or other physical measures or intervention activity.

As yet another example, an alert can be sent to the appropriate governmental authority. For example, if the disease is a foodborne illness, the computing system can alert a food or restaurant inspection authority.

In some implementations, the computing system will identify a particular location as being associated with a disease and/or send an alert regarding such location only if the particular location has experienced greater than a threshold number of visitors within a threshold amount of time. That is, if a location has only received a handful of visitors, it may not be statistically appropriate to determine that such location is associated with a disease based on the low number of users that provided disease-relevant information.

According to another aspect of the present disclosure, in some implementations, the computing system can determine or otherwise obtain predicted future location data for users. The predicted future location data can describe predicted future locations of the users. For example, the predicted future locations can be predicted on the basis of location patterns (e.g., commuting habits), calendar appointments, or other location signals or user data that indicate a future location for the user. The computing system can obtain the predicted future location data for all users or just for the second plurality of users. Further, as described above, the computing system can remove personal data from this user data and/or future location data so that only the location information remains without the corresponding personal information such as specific user identifiers.

In some implementations, the computing system can identify one or more locations predicted to be associated with the disease in the future based at least in part on the predicted future location data respectively associated with at least the second plurality of users. For example, if a significant number (e.g., one or more) of the second plurality of users are expected to visit a certain location at a certain time, then such location can be identified as being potentially associated with the disease in the future. As a result, the computing system can take preventative measures such as preemptively triggering a disease abatement system and/or notifying users regarding this location that is potentially associated with the disease in the future. As one example, the computing system can provide navigational instructions to a user that avoid the one or more locations predicted to be associated with the disease in the future, such that the user does not visit the location(s). For example, the computing system can provide navigational instructions which advise the user to take a certain transit vehicle (e.g., the 6:30 train) rather than some other transit vehicle (e.g., the 5:30 train) based a prediction that the other transit vehicle will be associated with elevated levels of the disease (e.g., have a large number of riders suffering from the flu).

The computing system can also provide navigational instructions which instruct the user of a specific route that avoids the locations predicted to be associated with elevated levels of the disease. This route may be a walking route, a bicycle route or a driving route. The system may also provide real-time, route-guidance information to the user in dependence on the user's real-world position where such route-guidance information uses a route avoiding the locations detected. Furthermore, the route may be updated in real-time as more locations associated with elevated levels of the disease are detected. Navigational instructions may also be supplied to the control system of an autonomous vehicle, e.g. a self-driving car. The autonomous vehicle can then transit along a route that does not contain, or at least has a reduced number of, locations where an elevated risk of the disease has been detected.

According to another aspect of the present disclosure, in some implementations, the machine-learned disease detection model can be trained using a supervised or semi-supervised learning technique. In one example, the disease detection model can be trained on data (e.g., search engine data) that has been labelled with automatically inferred labels, thereby preserving privacy and enabling deployment at scale. In particular, the computing system can leverage the fact that queries that lead to significant time spent on web pages about a particular disease (broadly defined including disease treatments and symptoms) are more likely to be about such particular disease. For example, certain web pages that correspond to certain diseases can be manually or automatically identified, labeled, and then used to automatically infer that a query corresponds to a particular disease when a user visits one of the web pages after entering the query. Anchoring on web pages allows the system to regularize over the noise in the raw queries, which—unlike pages—tend to be short, ambiguous, and sometimes full of typos.

In some implementations, the training pipeline collects queries leading to these web sites, and uses them as positive examples. Then, it samples other queries to serve as negative examples. Finally, the disease detection model can be trained using these two (automatically labelled) sets of queries. As a result, web page annotations are much more robust than query annotations. This approach can be used to train the machine-learned disease detection model.

Furthermore, in some implementations, the training data and/or the trained model can be validated or otherwise curated based on a manual review of the training data by medical experts. For example, one or more medical experts can review the search engine data and provide a medical opinion about whether the user is suffering from a particular disease. This medical opinion can be used to create additional training data, refine and/or validate existing training data, and/or validate the model performance based on new examples. For example, the medical experts' opinion can be used to label example sets of search engine data as either positive or negative training examples for certain diseases.

According to another aspect of the present disclosure, in some implementations, some or all of the operations described above (e.g., removal of personal information, use of the machine-learned disease detection model, and/or identification of locations) can be performed as a single machine job. By performing such operations as a single machine job, all intermediate data is kept at the single machine and is not sent over a communication network, which may be more vulnerable to malicious activities. Further, the intermediate data is not required to be stored in a database but instead can only be temporarily held at the single machine and then deleted upon completion of the processing. Thus, in some implementations, user privacy can be improved by performing all processing as a single machine job.

The systems and methods of the present disclosure provide a number of technical effects and benefits. As one example, the computing systems described herein can work in real time to detect locations experiencing elevated levels of a disease. In particular, the computing systems of the present disclosure complement existing data and approaches with real time signals available at scale. By working in real time based on online data, the systems and methods of the present disclosure can lead to early identification and, therefore, early or preemptive abatement of diseases. Early or preemptive abatement of diseases can lead to fewer numbers of persons acquiring or otherwise suffering from the disease, which corresponds to drastic improvements in public health. In particular, due to the exponential nature of transmission of certain diseases (e.g., highly infectious diseases), early or preemptive abatement of diseases can have a tremendously successful impact in preventing the spread of the disease.

A reduction in disease incidence and spread can be demonstrated in a number of different ways. As one example, AB testing on users receiving health notifications versus not longer-term reduction of official disease can be used. As another example, a reduction in disease incidence can be measured by official disease tracking organizations such as the Centers for Disease Control and Prevention and/or the World Health Organization. In some instances, the reduction may be difficult to observe or prove on country-level data. In such instances, notifications can be strategically deployed to select locations to be able to statistically demonstrate the difference. Finer granularity than country-level can also be evaluated.

The described systems and methods may also allow disease abatement to be efficiently performed by autonomous devices (e.g. mosquito spraying vehicles) which can be automatically dispatched and directed by these systems to the detected locations. Thus, one or more autonomous vehicles can be controlled based on the one or more identified locations.

Similarly, stationary ‘smart’ devices (e.g. an air filtration system) can be activated at the detected locations on receiving said information. Using such information may also allow for a significant proportion of the health benefits provided by the use of these devices to be obtained with reduced resource usage. For example, an air filtration system may consume significant quantities of energy so may be uneconomical to run continuously. Therefore, the described computing systems and methods may be used to selectively activate the air filtration system, or other similar systems, when an elevated risk of a relevant disease (e.g. an allergic disease) is detected at that location. Thus, various device(s) can be activated for reducing the incidence of the disease at at least one of the one or more identified locations associated with the disease. As another technical effect and benefit, the systems and methods of the present disclosure provide a streamlined process which can operate based on real time online data such as search engine data, which is already collected for a number of different purposes. Thus, the systems and methods of the present disclosure reduce the need for additional, objective-specific data collection such as government-collected statistics that are several years delayed, aggregated, expensive to curate, and cover only limited jurisdictions. Therefore, the systems and methods of the present disclosure can operate at a reduced cost relative to existing systems which require extensive survey collection and/or data maintenance. Further, the systems and methods of the present disclosure can operate at a significantly larger scale than certain existing systems that rely on localized surveys or other small-scale data sources.

As another technical effect and benefit, the systems and methods described herein, and particularly the use of machine-learned models, enables improved results with reduced computing resource expenditure. For example, by accurately predicting outbreaks from a relatively small quantity of targeted user data (e.g., specifically from search engine data rather than all available data), the systems and methods described herein can reduce the amount of processing and storage resources needed to identify disease outbreaks. Likewise, bandwidth or network usage can reduced by performing all operations as a single machine job.

With reference now to the Figures, example embodiments of the present disclosure will be discussed in further detail.

### Example Devices and Systems

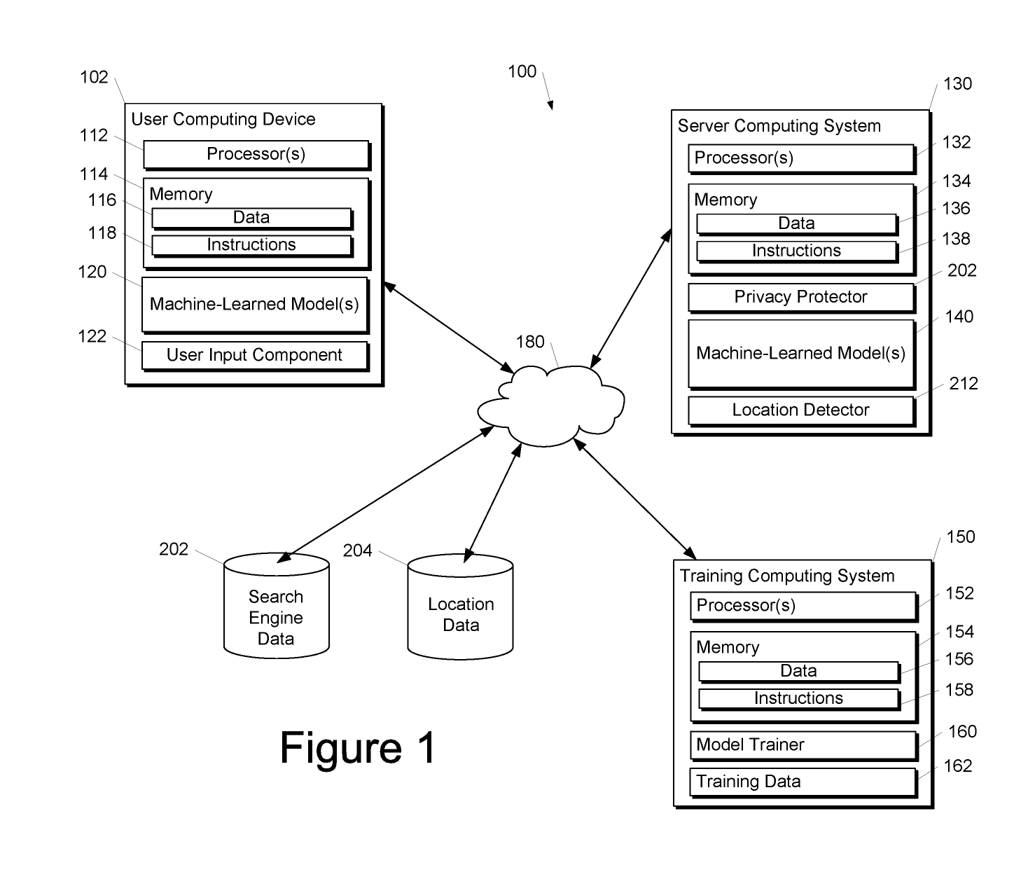

FIG. 1 depicts a block diagram of an example computing system 100 that performs disease tracking, monitoring, and prevention according to example embodiments of the present disclosure. The system 100 is provided as one example only. Other computing systems that include different components can be used in addition or alternatively to the system 100.

The system 100 includes a user computing device 102, a server computing system 130, and a training computing system 150 that are communicatively coupled over a network 180.

The user computing device 102 can be any type of computing device, such as, for example, a personal computing device (e.g., laptop or desktop), a mobile computing device (e.g., smartphone or tablet), a gaming console or controller, a wearable computing device, an embedded computing device, or any other type of computing device.

The user computing device 102 includes one or more processors 112 and a memory 114. The one or more processors 112 can be any suitable processing device (e.g., a processor core, a microprocessor, an ASIC, a FPGA, a controller, a microcontroller, etc.) and can be one processor or a plurality of processors that are operatively connected. The memory 114 can include one or more non-transitory computer-readable storage mediums, such as RAM, ROM, EEPROM, EPROM, flash memory devices, magnetic disks, etc., and combinations thereof. The memory 114 can store data 116 and instructions 118 which are executed by the processor 112 to cause the user computing device 102 to perform operations.

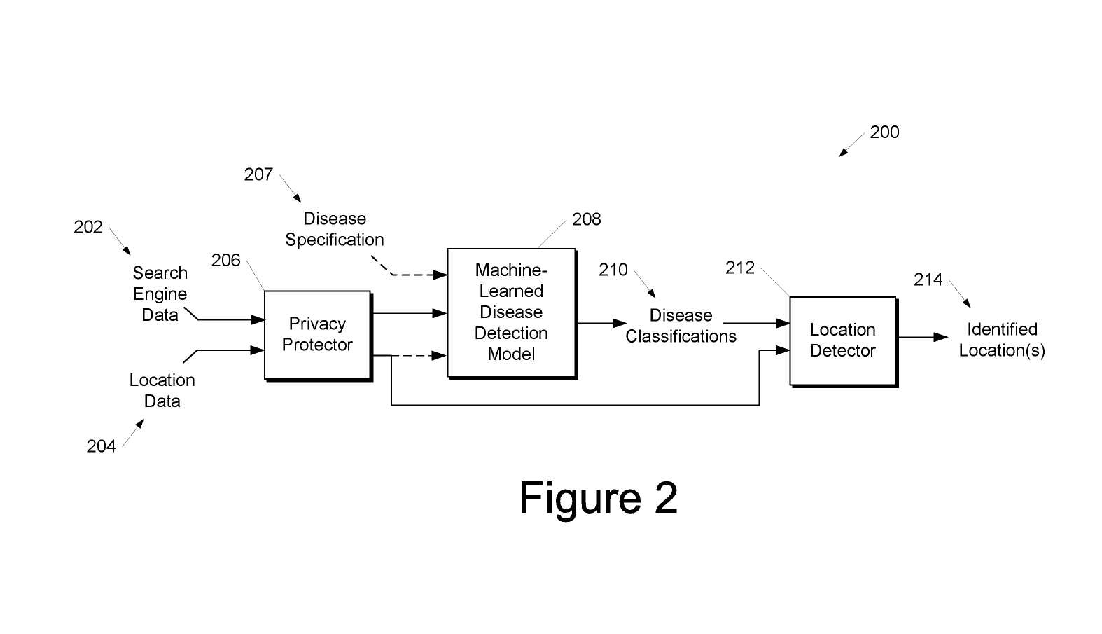

In some implementations, the user computing device 102 can store or include one or more machine-learned models 120. For example, the machine-learned models 120 can be or can otherwise include various machine-learned models such as support vector machines, log linear models, neural networks (e.g., deep neural networks), and/or other types of machine-learned models, including non-linear models and/or linear models. Neural networks can include feed-forward neural networks, recurrent neural networks (e.g., long short-term memory recurrent neural networks), convolutional neural networks or other forms of neural networks. Example machine-learned models 120 are discussed with reference to FIG. 2.

In some implementations, the one or more machine-learned models 120 can be received from the server computing system 130 over network 180, stored in the user computing device memory 114, and then used or otherwise implemented by the one or more processors 112.

Additionally or alternatively, one or more machine-learned models 140 can be included in or otherwise stored and implemented by the server computing system 130 that communicates with the user computing device 102 according to a client-server relationship. For example, the machine-learned models 140 can be implemented by the server computing system 140 as a portion of a web service. Thus, one or more models 120 can be stored and implemented at the user computing device 102 and/or one or more models 140 can be stored and implemented at the server computing system 130.

The user computing device 102 can also include one or more user input component 122 that receives user input. For example, the user input component 122 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

The server computing system 130 includes one or more processors 132 and a memory 134. The one or more processors 132 can be any suitable processing device (e.g., a processor core, a microprocessor, an ASIC, a FPGA, a controller, a microcontroller, etc.) and can be one processor or a plurality of processors that are operatively connected. The memory 134 can include one or more non-transitory computer-readable storage mediums, such as RAM, ROM, EEPROM, EPROM, flash memory devices, magnetic disks, etc., and combinations thereof. The memory 134 can store data 136 and instructions 138 which are executed by the processor 132 to cause the server computing system 130 to perform operations.

In some implementations, the server computing system 130 includes or is otherwise implemented by one or more server computing devices. In instances in which the server computing system 130 includes plural server computing devices, such server computing devices can operate according to sequential computing architectures, parallel computing architectures, or some combination thereof

As described above, the server computing system 130 can store or otherwise include one or more machine-learned models 140. For example, the models 140 can be or can otherwise include various machine-learned models. Example machine-learned models include support vector machines, log linear models, neural networks (e.g., deep neural networks), and/or other forms of multi-layer non-linear models. Example neural networks include feed forward neural networks, deep neural networks, recurrent neural networks, and convolutional neural networks. Example models 140 are discussed with reference to FIG. 2.

The server computing system 130 can also include a privacy protector 202 and a location detector 212. The privacy protector 202 and the location detector 212 will be discussed further with reference to FIG. 2.

The user computing device 102 and/or the server computing system 130 can train the models 120 and/or 140 via interaction with the training computing system 150 that is communicatively coupled over the network 180. The training computing system 150 can be separate from the server computing system 130 or can be a portion of the server computing system 130.

The training computing system 150 includes one or more processors 152 and a memory 154. The one or more processors 152 can be any suitable processing device (e.g., a processor core, a microprocessor, an ASIC, a FPGA, a controller, a microcontroller, etc.) and can be one processor or a plurality of processors that are operatively connected. The memory 154 can include one or more non-transitory computer-readable storage mediums, such as RAM, ROM, EEPROM, EPROM, flash memory devices, magnetic disks, etc., and combinations thereof. The memory 154 can store data 156 and instructions 158 which are executed by the processor 152 to cause the training computing system 150 to perform operations. In some implementations, the training computing system 150 includes or is otherwise implemented by one or more server computing devices.

The training computing system 150 can include a model trainer 160 that trains the machine-learned models 120 and/or 140 stored at the user computing device 102 and/or the server computing system 130 using various training or learning techniques, such as, for example, backwards propagation of errors. In some implementations, performing backwards propagation of errors can include performing truncated backpropagation through time. The model trainer 160 can perform a number of generalization techniques (e.g., weight decays, dropouts, etc.) to improve the generalization capability of the models being trained.

In particular, the model trainer 160 can train the machine-learned models 120 and/or 140 based on a set of training data 142. The training data 142 can include, for example, data (e.g., search engine data) that has been labelled with automatically inferred labels, thereby preserving privacy and enabling deployment at scale. In particular, the computing system can leverage the fact that queries that lead to significant time spent on web pages about a particular disease (broadly defined including disease treatments and symptoms) are more likely to be about such particular disease. For example, certain web pages that correspond to certain diseases can be manually or automatically identified, labeled, and then used to automatically infer whether a query corresponds to a particular disease. Anchoring on web pages allows the system to regularize over the noise in the raw queries, which—unlike pages—tend to be short, ambiguous, and sometimes full of typos.

In some implementations, the training pipeline collects queries leading to these web sites, and uses them as positive examples. Then, it samples other queries to serve as negative examples. Finally, the disease detection model can be trained using these two sets of queries. As a result, web page annotations are much more robust than query annotations. This approach can be used to train the machine-learned disease detection model.

Furthermore, in some implementations, the training data 142 and/or the trained model can be validated or otherwise curated based on a manual review of the training data 142 by medical experts. For example, one or more medical experts can review the search engine data and provide a medical opinion about whether the user is suffering from a particular disease. This medical opinion can be used to create additional training data, refine and/or validate existing training data, and/or validate the model performance based on new examples. For example, the medical experts' opinion can be used to label example sets of search engine data as either positive or negative training examples for certain diseases.

In some implementations, if the user has provided consent, the training examples can be provided by the user computing device 102. Thus, in such implementations, the model 120 provided to the user computing device 102 can be trained by the training computing system 150 on user-specific data received from the user computing device 102. In some instances, this process can be referred to as personalizing the model.

The model trainer 160 includes computer logic utilized to provide desired functionality. The model trainer 160 can be implemented in hardware, firmware, and/or software controlling a general purpose processor. For example, in some implementations, the model trainer 160 includes program files stored on a storage device, loaded into a memory and executed by one or more processors. In other implementations, the model trainer 160 includes one or more sets of computer-executable instructions that are stored in a tangible computer-readable storage medium such as RAM hard disk or optical or magnetic media.

The network 180 can be any type of communications network, such as a local area network (e.g., intranet), wide area network (e.g., Internet), or some combination thereof and can include any number of wired or wireless links. In general, communication over the network 180 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

FIG. 1 illustrates one example computing system that can be used to implement the present disclosure. Other computing systems can be used as well. For example, in some implementations, the user computing device 102 can include the model trainer 160 and the training dataset 162. In such implementations, the models 120 can be both trained and used locally at the user computing device 102. In some of such implementations, the user computing device 102 can implement the model trainer 160 to personalize the models 120 based on user-specific data.

Any components illustrated as being included in one of device 102, system 130, and/or system 150 can instead be included at one or both of the others of device 102, system 130, and/or system 150. For example, the privacy protector 202 and/or the location detector 212 can be included in the user computing device 102.

### Example Processing Pipeline

FIG. 2 depicts a block diagram of an example processing pipeline 200 according to example embodiments of the present disclosure. The pipeline 200 can include a privacy protector 206, a machine-learned disease detection model 208, and a location detector 212. The pipeline 200 can process data to identify locations associated with an elevated level of a disease.

The privacy protector 206 can receive search engine data 202 and location data 204 associated with each of a first plurality of users. For example, the location data 204 can include current location data that describes current location of users and/or location history data that describes historical locations of users in the past.

In some implementations, the privacy protector 206 can obtain the search engine data 202 and/or location data 204 from or in the form of logs stored in one or more databases associated with a search engine service and/or location service. As examples, the search engine data 202 can include data descriptive of search queries; scrolling actions; result selection actions; time spent on user-selected results; content of user-selected results; and/or other search engine information.

The privacy protector 206 can remove personal information from the search engine data 202 and the location data 204. As one example, the privacy protector 206 can respectively assign a unique and non-personally identifying identifier to the search engine data 202 for each of the first plurality of users and then remove any personal information from the search engine data 202. As examples, the unique and non-personally identifying identifier can include a random number, a result of a hash function applied to a user identifier, or some other obfuscated or encrypted identifier which cannot be reverted to obtain the user identifier. Likewise, the privacy protector 206 can respectively assign the same unique and non-personally identifying identifier to the location data 204 for each of the first plurality of users and then remove personal information from the location data 204. Thus, the privacy protector 206 can output sets of search engine data that are respectively connectable to sets of location data, but which contain no personal information such as specific user identities.

The machine-learned disease detection model 208 can receive the privacy protected search engine data 202 and, optionally, also the privacy protected location data 204. In some implementations, the machine-learned disease detection model 208 can also receive any additional feature data (not shown).

The first plurality of users for which the machine-learned disease detection model 208 receives the search engine data 202 and, optionally, also the location data 204 can include any number of different users. As one example, the first plurality of users can include all users for which search engine data is available (e.g., all users globally). In another example, the first plurality of users can be limited to users that have a particular characteristic. For example, the first plurality of users can be limited to users that reside in or are otherwise associated with a particular location (e.g., limited to users that reside or are otherwise located in a particular country, state, county, and/or city). In another particular example, the first plurality of users can be limited to users that have visited a particular location (e.g., an evacuation shelter). In yet another example, the first plurality of users can be limited to users that have search engine data structured according to a particular language (e.g., users that use English-language search queries).

In some implementations, the machine-learned disease detection model 208 can also receive a disease specification 207. The disease specification 207 can specify a particular disease for which the model 208 should screen. As one example, the disease specification 207 can be or take the form of a unique identifier associated with the disease. For example, the unique identifier can be a KG mid or ICD code. In some implementations, the model 208 can use the disease specification 207 to lookup an incubation period and/or other aspects of the disease. Alternatively, such information can be included in the specification 207. In some implementations, through use of the disease specification 207, the model 208 can be altered to look for various diseases including, as examples, foodborne diseases, vector borne diseases (e.g., malaria, dengue, etc.), and/or environment conditions (e.g., pollution, allergy, etc.).

The machine-learned disease detection model 208 can output disease classifications 210. The disease classifications 210 can indicate which of the first plurality of users are predicted to have the disease.

In some implementations, the machine-learned disease detection model 208 can operate to provide a prediction on a per-search query basis. For example, the machine-learned disease detection model 208 can provide, for each search query, a probability that the search query is about the disease. For example, the probability can be a score between 0 and 1 for each query.

In some implementations, a user that submitted the search query can be predicted to have the disease with the same or different probability. For example, a user that has submitted multiple queries can be predicted to have the disease with a probability equal to or based on a sum of the probabilities assigned to her multiple queries and/or a weighted average of the probabilities assigned to her multiple queries.

In other implementations, the machine-learned disease detection model 208 can operate to provide a prediction on a per-user basis. For example, the machine-learned disease detection model 208 can obtain all search engine data associated with a user and output a single probability that the user is suffering from the disease.

In some implementations, predictions can be binarized using a probability threshold. For example, the probability associated with a prediction (e.g., a user-level prediction and/or a query-level prediction) can be compared to the probability threshold to determine whether the prediction should be changed to a value of “1” (that is, confirmed) or changed to a value of “0” (that is, discarded or reversed). As one example, the probability threshold can be 0.93.

As examples, the machine-learned disease detection model 208 can be or include a support vector machine, a deep neural network, and/or a log-linear model. For example, the log-linear model can be a log-linear maximum entropy model.

In some implementations, the machine-learned disease detection model 208 can have a feature space of 50,000 dimensions and can leverage feature hashing for compactness. In some implementations, the features can include word unigrams and bigrams extracted from a query string, as well as from the search result URLs, snippets (e.g., short summaries of each result displayed by the search engine), web page titles, and/or other forms of search engine or online data as described above. In some implementations, features can also be constructed based on a knowledge graph (e.g., graph annotations of the concepts mentioned in the query).

In some implementations, certain queries and/or users can be filtered or otherwise removed. As one example, users who are unlikely to be suffering from the disease but are still querying for it can be removed. For example, these users can include users that may be researching the disease for academic purposes, searching for symptoms of a family member or a friend, or searching about a news story related to Lyme disease. As one example implementation of this concept, only users who issued three or more queries that the model 208 predicted as related to the disease can be predicted to be suffering from the disease.

The location detector 212 can receive the disease classification information 210 and the privacy protected location data 204 and can output one or more identified locations 214 associated with elevated levels of the disease. As one example, the location detector 212 can determine, for each location, a ratio of a first number users that visited the location and that are predicted to have the disease versus a second number of users that visited the location and that are not predicted to have the disease. As another example, the location detector 212 can determine, for each location, a ratio of a first number users that visited the location and that are predicted to have the disease versus a total number of users that visited the location regardless of disease status (e.g., inclusive of all visitors). If the ratio for a particular location exceeds a threshold value (e.g., a location-specific threshold value), then the location detector 212 can designate such location as being associated with elevated levels of the disease. As one example, a visitor to a location can include a resident of a location, an employee of a location, someone who visited the location only briefly, on other connections between a person and a location.

Each of the privacy protector 212 and the location detector 212 include computer logic utilized to provide desired functionality. Each of the privacy protector 212 and the location detector 212 can be implemented in hardware, firmware, and/or software controlling a general purpose processor. For example, in some implementations, each of the privacy protector 212 and the location detector 212 includes program files stored on a storage device, loaded into a memory and executed by one or more processors. In other implementations, each of the privacy protector 212 and the location detector 212 includes one or more sets of computer-executable instructions that are stored in a tangible computer-readable storage medium such as RAM hard disk or optical or magnetic media.

According to another aspect of the present disclosure, in some implementations, some or all of the processing pipeline 200 can be performed as a single machine job. By performing the pipeline 200 as a single machine job, all intermediate data (e.g., disease classifications 210) is kept at the single machine and is not sent over a communication network, which may be more vulnerable to malicious activities. Further, the intermediate data is not required to be stored in a database but instead can only be temporarily held at the single machine and then deleted upon completion of the processing (e.g., upon output of the identified locations 214). Thus, in some implementations, user privacy can be improved by performing all of the processing pipeline 200 as a single machine job.

According to another aspect of the present disclosure, in some implementations, the computing system that implements the processing pipeline 200 can further generate a displacement map or other data structure that indicates the movement or mobility of humans. For example, the displacement map can be generated by modeling aggregated location data (e.g., from user devices such as smartphones). As one example, the planet can be divided into cells (e.g., 1 square kilometer in size). The computing system can calculate, for each pair of cells, how many people travel from cell A to cell B, thereby generating a displacement map. As a result, the displacement map can quantify the flow of people from one area to another. The displacement map can be combined with the identified locations 214 associated with elevated levels of a disease to identify additional locations where the disease may be migrating or otherwise moving. For example, if cell A is identified as currently experiencing an elevated level of a disease and the displacement map indicates a large amount of movement from cell A to cell B, then steps can be taken to combat the disease at cell B.

### Example Methods

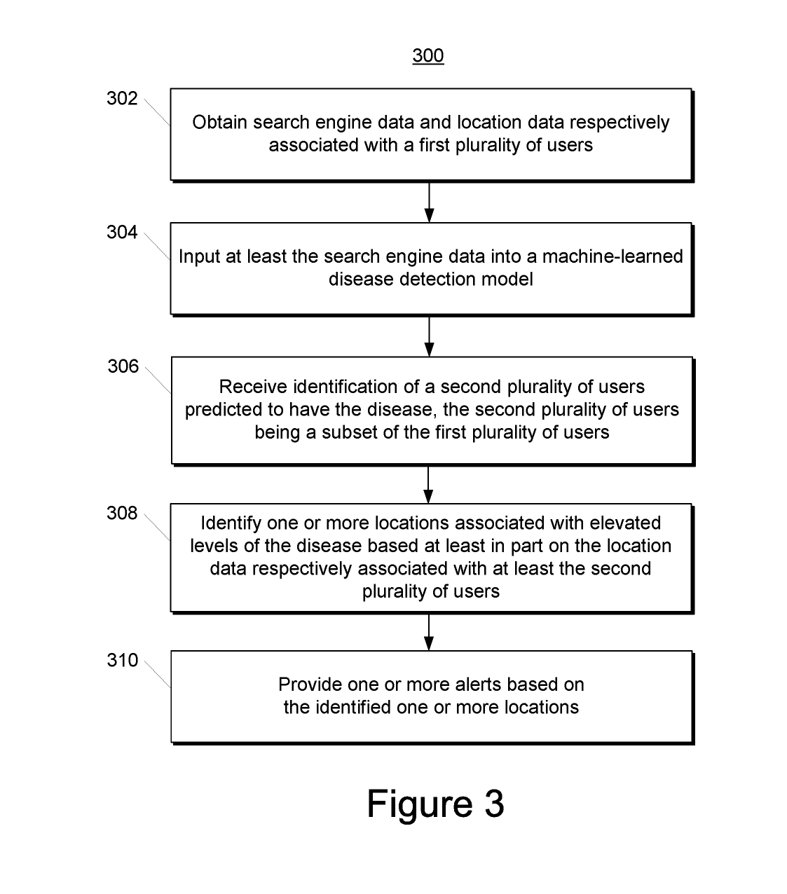

FIG. 3 depicts a flow chart diagram of an example method to perform according to example embodiments of the present disclosure. Although FIG. 3 depicts steps performed in a particular order for purposes of illustration and discussion, the methods of the present disclosure are not limited to the particularly illustrated order or arrangement. The various steps of the method 300 can be omitted, rearranged, combined, and/or adapted in various ways without deviating from the scope of the present disclosure.

At 302, a computing system can obtain search engine data and location data (e.g., location history data) respectively associated with a first plurality of users. For example, the location data can include current location data that describes current location of users and/or location history data that describes historical locations of users in the past.

In some implementations, the computing system can obtain the search engine data and/or location data from or in the form of logs stored in one or more databases associated with a search engine service and/or location service. As examples, the search engine data can include data descriptive of search queries; scrolling actions; result selection actions; time spent on user-selected results; content of user-selected results; and/or other search engine information.

According to another aspect of the present disclosure, in some implementations, the computing system can remove personal information from the obtained data so that information such as specific user identities is not included in the data. For example, in some implementations, the computing system can respectively assign a unique and non-personally identifying identifier to the search engine data for each of the first plurality of users and then remove any personal information from the search engine data. As examples, the unique and non-personally identifying identifier can include a random number, a result of a hash function applied to a user identifier, or some other obfuscated or encrypted identifier which cannot be reverted to obtain the user identifier. Likewise, the computing system can respectively assign the same unique and non-personally identifying identifier to the location history data for each of the first plurality of users and then remove personal information from the location data. Thus, the computing system can obtain sets of search engine data that are respectively connectable to sets of location history data, but which contain no personal information such as specific user identities.

At 304, the computing system can input at least the search engine data (and optionally also the location data) into a machine-learned disease detection model. For example, the computing system can input each set of search engine data for each user into the model individually (e.g., on a per-user basis). In some implementations, in addition or alternatively to the search engine data, the computing system can input the location data (e.g., location history data) into the machine-learned disease detection model. In some implementations, additional feature data can also be input into the machine-learned disease detection model. As examples, the additional feature data can include step count data for a user (e.g., that is generated by a computing device that is worn by the user); heart rate data for a user (e.g., that is generated by a computing device that is worn by the user); weather condition data (e.g., current and/or historical weather condition data such as temperature, humidity, etc. for a number of different location); mosquito density data; water quality data; and/or various other types of features including any features that have been shown to be predictors of and/or correlated to the disease being investigated.

At 306, the computing system can receive identification of a second plurality of users predicted to have the disease. The second plurality of users being a subset of the first plurality of users. Thus, in some implementations, the computing system can input the search engine data, location data (e.g., location history data), and/or other feature data associated with a user into the machine-learned disease detection model and can receive a prediction from the machine-learned disease detection model as to whether such user is suffering from the disease. In some implementations, the prediction can indicate whether the user is currently suffering from the disease and/or whether the user suffered from the disease in the past and, if so, at what time in the past.

As examples, the machine-learned disease detection model can be or include one or more of a support vector machine, a deep neural network, a log linear model, and/or other machine-learned models. In some implementations, the computing system can include multiple different machine-learned disease detection models that have been respectively trained to detect multiple different diseases (e.g., a foodborne illness model, a malaria model, etc.). In other implementations, a single machine-learned model can be trained to provide a multi-class classification prediction with respect to the multiple different diseases.

At 308, the computing system can identify one or more locations associated with elevated levels of the disease based at least in part on the location data (e.g., location history data) respectively associated with at least the second plurality of users. Thus, for example, the computing system can analyze current and/or past locations associated with the second plurality of users to identify locations that exhibit an increased or otherwise elevated number of persons with the disease.

In some implementations, the computing system can identify the one or more locations associated with elevated levels of the disease based at least in part on the location data (e.g., location history data) respectively associated with both the second plurality of users and a third plurality of users. The third plurality of users can include those users that are included in the first plurality of users but not the second plurality of users.

As one example, in some implementations, the computing system can identify the one or more locations from a plurality of candidate locations. For example, the candidate locations can be geographical areas (e.g., map tiles that correspond to certain areas of the Earth, states, counties, cities, neighborhoods, property parcels, or other divisions of geographic area); establishments (e.g., business storefronts such as restaurants); buildings; geographic coordinates; or other representations of location.

In some implementations, to identify the one or more locations associated with the disease from the one or more candidate locations, the computing system can determining, for each of the plurality of candidate locations, a ratio of a first number of the second plurality of users that have visited the candidate location within a threshold amount of time to a second number of the third plurality of users that have visited the candidate location within the threshold amount of time.

The computing system can identify one or more of the candidate locations as being associated with the disease based at least in part on the respective ratios determined for the plurality of candidate locations. As one example, the computing system can compare each respective ratio to a threshold value to determine whether the corresponding candidate location is associated with the disease. For example, if the ratio is greater than the threshold value, then the candidate location can be designated as being associated with elevated levels of the disease.

In some implementations, the threshold value used for each candidate location can be different or otherwise specific to such candidate location. For example, the threshold value for a candidate location can be based on a baseline ratio that has been demonstrated at such candidate location in the past.

As another example, in some implementations, the computing system can identify the one or more locations associated with the disease by inputting the location history data respectively associated with the second plurality of users into a machine-learned location detection model. The computing system can receive identification of one or more visited locations associated with the second plurality of users. In some implementations, the visited locations can be designated as associated with the disease. However, in other implementations, the visited locations identified by the machine-learned location detection model can simply be used to compute the ratios described above for various locations. As such, in some implementations, the computing system can input the location history data for all users (e.g., including the third plurality of users) to simply receive indications of locations that each user has visited (e.g., for the purpose of computing ratios as described above).

At 310, the computing system can provide one or more alerts based on the identified one or more locations. For example, the computing system can provide one or more alerts to users, disease abatement systems, and/or appropriate governmental authorities regarding the locations that have been identified as having elevated levels of the disease. As one example, an alert can be sent to a user (e.g., a user that regularly visits or is planning to visit the location) that informs the user to avoid the location.

As another example, an alert can be sent to a disease abatement system. For example, if the disease is a mosquito-borne illness, the computing system can alert (e.g., automatically dispatch) a mosquito spraying vehicle to spray the identified locations.

As yet another example, an alert can be sent to the appropriate governmental authority. For example, if the disease is a foodborne illness, the computing system can alert a food or restaurant inspection authority.

In some implementations, the computing system will identify a particular location as being associated with a disease and/or send an alert regarding such location only if the particular location has experienced greater than a threshold number of visitors within a threshold amount of time. That is, if a location has only received a handful of visitors, it may not be statistically appropriate to determine that such location is associated with a disease based on the low number of users that provided disease-relevant information.

### Additional Disclosure

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

What is claimed is:

1. A computing system, comprising:
one or more processors;
a machine-learned disease detection model; and
one or more non-transitory computer-readable media that collectively store instructions that, when executed by the one or more processors, cause the one or more processors to perform operations, the operations comprising:
obtaining search engine data and location history data respectively associated with a first plurality of users;
inputting at least the search engine data into a machine-learned disease detection model;
receiving, as an output of the machine-learned disease detection model, identification of a second plurality of users predicted to have the disease, the second plurality of users being a subset of the first plurality of users; and
identifying one or more locations associated with elevated levels of the disease based at least in part on the location history data respectively associated with at least the second plurality of users.

2. The computing system of claim 1, wherein the operations further comprise, prior to said inputting, receiving, and identifying:
respectively assigning a unique and non-personally identifying identifier to the search engine data for each of the first plurality of users;
respectively assigning the same unique and non-personally identifying identifier to the location history data for each of the first plurality of users; and
removing personal information from the search engine data and the location history data.

3. The computing system of claim 1, wherein the search engine data comprises data descriptive of one or more of:
search queries;
scrolling actions;
result selection actions;
time spent on user-selected results; or
content of user-selected results.

4. The computing system of claim 1, wherein inputting at least the search engine data into the machine-learned disease detection model comprises inputting both the search engine data and the location history data into the machine-learned disease detection model.

5. The computing system of claim 1, wherein inputting at least the search engine data into the machine-learned disease detection model comprises inputting both the search engine data and additional feature data into the machine-learned disease detection model, wherein the additional feature data comprises one or more of:
step count data generated by a computing device that is worn;
heart rate data generated by a computing device that is worn;
weather condition data;
mosquito density data; or
water quality data.

6. The computing system of claim 1, wherein identifying the one or more locations associated with elevated levels of the disease comprises identifying the one or more locations associated with elevated levels of the disease based at least in part on the location history data respectively associated with both the second plurality of users and a third plurality of users, the third plurality of users comprising those users included in the first plurality of users but not the second plurality of users.

7. The computing system of claim 6, wherein identifying the one or more locations associated with elevated levels of the disease comprises:
identifying a plurality of candidate locations;
determining, for each of the plurality of candidate locations, a ratio of a first number of the second plurality of users that have visited the candidate location within a threshold amount of time to a second number of the third plurality of users that have visited the candidate location within the threshold amount of time; and
identifying one or more of the candidate locations as being associated with the disease based at least in part on the respective ratios determined for the plurality of candidate locations.

8. The computing system of claim 7, wherein identifying the one or more of the candidate locations as being associated with the disease based at least in part on the respective ratios comprises comparing each respective ratio to a threshold value.

9. The computing system of claim 7, wherein the disease comprises foodborne illness and the plurality of candidate locations comprises a plurality of restaurants.

10. The computing system of claim 7, wherein the plurality of candidate locations comprises a plurality of geographic areas.

11. The computing system of claim 1, wherein identifying the one or more locations associated with elevated levels of the disease comprises:
inputting, by the one or more computing devices, the location history data respectively associated with the second plurality of users into a machine-learned location detection model; and
receiving, by the one or more computing devices, identification of one or more visited locations associated with the second plurality of users.

12. The computing system of claim 1, further comprising:
determining, for each of the one or more locations a number of visitors to such location within a threshold amount of time; and
for each of the one or more locations for which the number of visitors to such location exceeds a threshold number of visitors, providing an alert regarding the disease at such location.

13. The computing system of claim 1, further comprising:
automatically dispatching a disease abatement tool to the one or more locations associated with elevated levels of the disease.

14. The computer-implemented method of claim 1, wherein the machine-learned disease detection model comprises one or more of: a support vector machine; a deep neural network; or a log linear model.

15. A computer-implemented method to identify locations associated with a disease, the method comprising:
obtaining, by one or more computing devices, search engine data and location data respectively associated with a first plurality of users;
inputting, by the one or more computing devices, at least the search engine data into a machine-learned disease detection model;
receiving, by the one or more computing devices as an output of the machine-learned disease detection model, identification of a second plurality of users predicted to have the disease, the second plurality of users being a subset of the first plurality of users;
identifying, by the one or more computing devices, one or more locations associated with the disease based at least in part on the location data respectively associated with at least the second plurality of users.

16. The computer-implemented method of claim 15, wherein inputting, by the one or more computing devices, at least the search engine data into the machine-learned disease detection model comprises inputting, by the one or more computing devices, both the search engine data and the location data into the machine-learned disease detection model.

17. The computer-implemented method of claim 15, wherein identifying, by the one or more computing devices, the one or more locations associated with the disease comprises identifying, by the one or more computing devices, the one or more locations associated with the disease based at least in part on the location data respectively associated with the second plurality of users and a third plurality of users, the third plurality of users comprising those users included in the first plurality of users but not the second plurality of users.

18. The computer-implemented method of claim 15, wherein:
the location data comprises predicted future location data; and
identifying, by the one or more computing devices, the one or more locations associated with the disease comprises identifying, by the one or more computing devices, one or more locations predicted to be associated with the disease in the future based at least in part on the predicted future location data respectively associated with at least the second plurality of users.

19. The computer-implemented method of claim 18, further comprising:
providing, by the one or more computing devices, navigational instructions that avoid the one or more locations predicted to be associated with the disease in the future.

20. One or more non-transitory computer-readable media that collectively store instructions that, when executed by one or more processors, cause the one or more processors to perform operations, the operations comprising:
obtaining location history data respectively associated with a first plurality of users;
inputting the location history data into a machine-learned disease detection model;
receiving, as an output of the machine-learned disease detection model, identification of a second plurality of users predicted to have the disease, the second plurality of users being a subset of the first plurality of users; and
identifying one or more locations associated with the disease based at least in part on the location history data respectively associated with at least the second plurality of users.

21. A computer-implemented method, the method comprising:
receiving, by one or more computing devices, user data;
determining, by the one or more computing devices, correlations between the user data and one or both of disease or location;
storing, by the one or more computing devices, the correlations;
iteratively updating, by the one or more computing devices, the correlations upon receipt of new user data to form a predictive disease detection model; and
using, by the one or more computing devices, the predictive disease detection model to determine a predicted causation of a disease outbreak.

22. The computer-implemented method of claim 21, wherein the predicted causation comprises a predicted location.
